(19) United States
(12) Patent Application Publication
(10) Pub. No.: US 2025/0057734 A1
(43) Pub. Date: Feb. 20, 2025

(54) DENTAL GROWTH STIMULANT AND TREATMENT SET

(71) Applicant: Heidrun HOFMANN, Markt Indersdorf (DE)

(72) Inventor: Heidrun HOFMANN, Markt Indersdorf (DE)

(21) Appl. No.: 18/718,001

(22) PCT Filed: Dec. 8, 2022
(86) PCT No.: PCT/IB2022/061941

§ 371 Date: Jun. 7, 2024

(30) Foreign Application Priority Data
Dec. 8, 2021 (DE) 10 2021 132 335.5

(51) Int. Cl. A61K 6/898; A61K 6/54; A61K 6/853; A61K 6/878

(52) U.S. Cl. CPC A61K 6/898; A61K 6/54; A61K 6/853; A61K 6/878

(57) ABSTRACT

The invention relates to a dental growth stimulant for regenerating a bone structure, in particular within a tooth pocket, characterized in that the growth stimulant comprises at least a hyaluronic acid or a hyaluronic acid derivative, in particular a hyaluronic acid salt, and wherein the growth stimulant additionally has a calcium compound and/or a zirconium compound.

The present invention relates to a dental growth stimulant according to the preamble of claim 1 and a treatment set according to the preamble of claim 8.

EP 2 682 136 B1 discloses a dental root canal filling material for use in a method of treating dental caries or pulpitis by regenerating dental tissue, comprising the steps of injecting the dental canal filling material into the root tip side of a dental canal which has undergone pulp extirpation or dental canal dilatation and cleaning an infected root canal.

From WO 2004/011 053 A1 as well as from Parveen Dahiya, Reet Kamal, Hyaluronic Acid: A Boon in Periodontal Therapy, North American Journal of Medical Sciences, May 2013, Volume 5, Issue 5, the use of hyaluronic acid to stimulate bone formation is known. Bone growth of approx. 2 mm was observed over several months in the case of dental treatment.

It is the object of the present invention to improve bone formation.

A dental growth stimulant according to the invention serves to regenerate a bone structure. In the context of the present invention, bone structure includes in particular both the jawbone and the human teeth.

The regeneration of the bone structure also includes the treatment of periodontitis, also known as paradontosis. The application can be carried out in particular in a tooth pocket, both in a tooth that has been treated for the first time and in a tooth or a tooth remnant on which several root canal treatments have already been carried out.

The aforementioned growth stimulant has at least one hyaluronic acid or a hyaluronic acid derivative, in particular a hyaluronic acid salt, and also a calcium compound and/or a zirconium compound.

Other components may also be included. Surprisingly, it was found that compared to a simple hyaluron application, growth approximately doubled to a bone build-up of 4 mm in 4 months with the previously known combination. As a result, the patient's periodontal status was significantly improved and the tooth could be preserved.

Advantageous embodiments of the invention are the subject matter of the dependent claims.

It is advantageous if the growth stimulant consists of the hyaluronic acid or the hyaluronic acid derivative, in particular the hyaluronic acid salt, and the calcium compound and/or the zirconium compound.

The calcium compound can advantageously be formed as a silicate compound, in particular as a tricalcium silicate.

The zirconium compound may be formed as zirconium (IV) oxide.

In particular, the dental growth stimulant may be for use in a method of treating paradontosis and/or building a bone by stimulating the bone in a tooth pocket comprising the steps of:
A refreshening the gums and/or bone; and
B dispensing the growth stimulant from a dosing device above a tooth pocket in such a way that the growth stimulant can fall into the tooth pocket due to gravity.

In particular, the dental growth stimulant may be for use in a method of treating paradontosis and/or building a bone by stimulating the bone in a tooth pocket comprising the steps of:
A performing a root canal treatment of a tooth with the hyaluronic acid or the hyaluronic acid derivative, in particular the hyaluronic acid salt, and
B dispensing the growth stimulant into a periodontal gap between the tooth and a jaw in which the tooth is located.

Anesthesia, in particular with a local anesthetic, can be administered prior to the refreshening or root canal treatment according to step A.

The growth stimulant can be multicomponent and formed by adding the individual components in situ at the site of application.

Further according to the invention is a treatment set comprising several storage packages for carrying out various steps of a dental treatment for bone formation and/or for the treatment of paradontosis, wherein the treatment set contains at least one storage package with the dental growth stimulant according to the invention or storage packages with several individual components for the in-situ formation of the dental growth stimulant according to the invention.

Advantageously, the treatment set may comprise a storage package of a local anesthetic.

Further advantageously, the treatment set may comprise a packaging housing, wherein the dosing packages are arranged in individual sterile packages in the packaging housing.

In the following, the procedure of a treatment with the growth stimulant according to the invention is explained in more detail using an example:

In this procedure, the gums are anesthetized, refreshened (i.e. bleeding is produced) and hyaluronic acid is added to the tooth pocket.

In addition, teeth that have already undergone root canal treatment are subjected to a renewal of the root treatment with a root canal filling (Gutta-Percha points) and Biosealer and/or Bioroot (bioactive tricalcium silicate from Septodent).

Bone growth occurs in approx. 80 percent of the cases. Ideally, up to 4 mm in 4 months.

In the example, the growth stimulant is formed in situ in the tooth pocket. However, it is also possible to form and apply the growth stimulant in advance as a mixture of the individual components.

The growth stimulant may also contain other agents, e.g. an antibiotic and/or a disinfectant to promote the healing of the re-bleeding wound. Hyaluronic acid or the hyaluronic acid derivative, and the calcium compound and/or zirconium compound advantageously form the main component of the growth stimulant, i.e. more than 50% by weight, preferably even more than 90% by weight.

The above-described properties, features and advantages of this invention, as well as the manner in which they are achieved, will become clearer in connection with the following description of the embodiments, which are explained in more detail in connection with the drawing, in which:

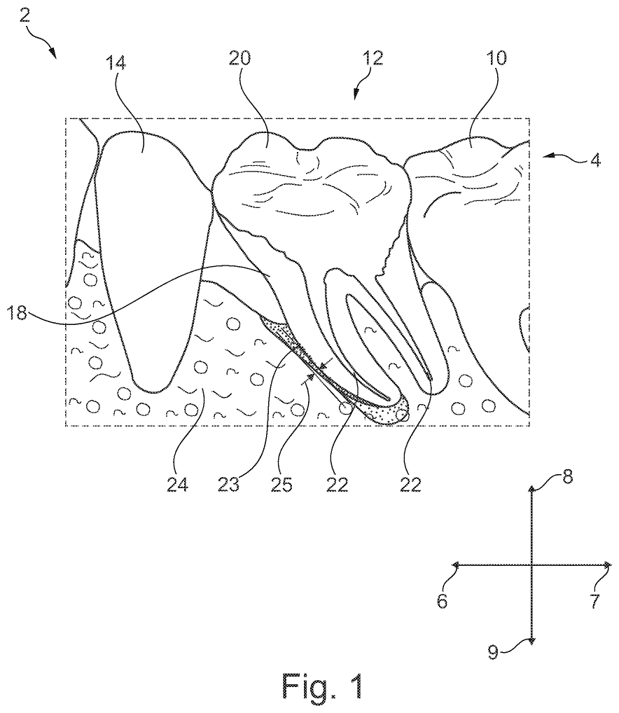

FIG. 1 shows a section of a human oral cavity in a region of the third quadrant of teeth, and

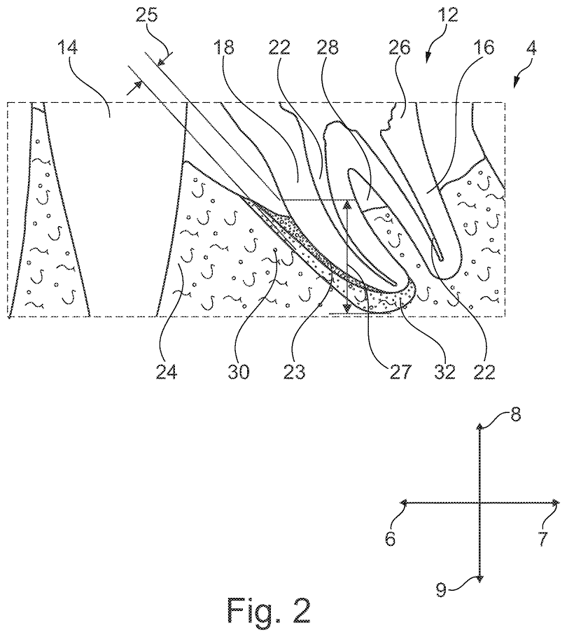

FIG. 2 shows a part of the section of FIG. 1.

In the figures, the same technical elements are provided with the same reference signs, and are only described once. The figures are purely schematic and, in particular, do not reflect the actual geometric proportions. Reference is made to FIG. 1, which shows a human oral cavity section 2 in an area of the third tooth quadrant 4. The oral cavity section 2 is viewed below in the directions mesial 6, distal 7, coronal 8 and apical 9.

The area of the third tooth quadrant 4 shown in FIG. 1 comprises a second molar tooth 10 distally 7 on the outer side, which is adjoined mesially 6 by a first molar tooth 12 and a premolar tooth 14.

In the following, only the first molar tooth 12 is considered further to explain the embodiment example. The first molar tooth 12 consists of a tooth root 16 starting apically 9, which is followed coronally 8 by a tooth neck 18 and again coronally 9 by a tooth crown 20. When seen in the apical direction 9 in the lower region of the tooth crown 20 and in the tooth neck 18, not further referenced pulp is included, consisting of blood vessels, nerve tracts, connective tissue and cells for the formation of dentin and supplying it with nutrients. To this end, root canals 22 run through the tooth root 16, which enable the dentin to be connected to a blood circulation system.

The first molar tooth 12 is embedded in the jawbone 24 via a periodontal gap 23. The periodontal gap 23 contains an elastic fiber system, the so-called Sharpey's fibers, which absorb the compressive forces acting on the first molar tooth 12, cushion them, transmit them to it and partially convert them into tensile forces. If the first molar tooth 12 is healthy, the periodontal gap 23 has a periodontal gap width 25 and a periodontal gap depth 27, which is assumed to be less than 3 mm for a healthy tooth. The basic structure of molar teeth is well known and will not be discussed further here. FIG. 1 shows the illustrated teeth 10, 12, 14 in a healthy condition. In contrast to this, part of the area of the third tooth quadrant 4 shown in FIG. 1 with teeth 10 and 12 is shown again in FIG. 2 below, but with the first molar tooth 12 in a condition requiring treatment.

First of all, the first molar tooth 12 has a cavity 26 caused by caries in the area between the tooth crown 20 and the tooth neck 18, which is, however, irrelevant for the further explanations.

In addition to the cavity 26 caused by caries, FIG. 2 also shows an apical whitening 32 seen coronally 8 below the tooth root 16 of the second molar tooth 12. This indicates a focus of inflammation which, if left untreated, leads to severe pain for the patient and, in the long term, to the loss of the second molar tooth 12.

Finally, the bone resorption on the first molar tooth 12, which is clearly visible in FIG. 2, is relevant for the present embodiment example. This can first be seen in an exposed furcation 28 in the area of the tooth root 16. This is difficult to access during oral hygiene and is therefore susceptible to germs, which is why it should be treated with periodontal surgery to prevent inflammation and further bone resorption. In addition to the exposed furcation 28, bone resorption can also be observed at the periodontal gap 23, which has a clearly recognizable enlarged periodontal gap depth 27. Finally, bone resorption can also be seen in the porous areas 30 of the jawbone 24.

The filling of the periodontal gap 23 with hyaluronic acid is known, for example, from WO 2004/011 053 A1 for the treatment of bone resorption. The periodontal gap 23 can be rejuvenated with such treatment using hyaluronic acid, but it cannot be closed. This is illustrated by the example shown in Table 1 of a patient whose open periodontal gap was treated with hyaluronic acid in the usual manner over a period of 6 months. Three measurements were taken. With the first measurement (measurement 1), the depth of the respective untreated and open periodontal gap was documented and treated with hyaluronic acid. With the second measurement (measurement 2), the depth of the respective periodontal gap was documented after five months and treated again with hyaluronic acid. After a further month, the depth of the respective periodontal gap was documented once again by means of the third measurement (measurement 3). The procedure described in P. Eickholz, Clinical Periodontal Diagnosis: Probing Pocket Depth, Vertical Attachment Level and Bleeding on Probing, 2004, Medicine, was used for the measurements. In Table 1, the tooth scheme of the Federation Dentaire Internationale (FDI) is used to identify the teeth.

TABLE 1

| Tooth FDI | Measurement 1 | Measurement 2 | Measurement 3 |
|---|---|---|---|
| 15 mesial | 6.8 mm | 5.87 mm | 5.4 mm |
| 15 distal | 10.3 mm | 7.94 mm | 7.14 mm |
| 17 mesial | 9.08 mm | 6.81 mm | 5.04 mm |
| 17 distal | 6.68 mm | 7.58 mm | 7.67 mm |

The measurement results show that although hyaluronic acid is generally able to reduce the size of the periodontal gap, it is not usually possible to close it completely—i.e. reduce its depth to less than 3 mm. It has also been shown that the use of hyaluronic acid alone is time-consuming and therefore results in a long treatment period for the patient.

In order to increase the treatment effect and also to reduce the duration of treatment, the invention combines hyaluronic acid in a dental growth stimulant for regenerating a bone structure, in this case within a tooth pocket, with a calcium compound and/or a zirconium compound. As an alternative to hyaluronic acid, a hyaluronic acid derivative, such as a hyaluronic acid salt, can also be used. Other components may also be included. It was a surprise to find out that growth can be more than doubled compared to an application with simple hyaluronic acid. It is possible to realize a bone formation of 4 mm in just 4 months. This means that significantly more teeth can be preserved in patients in periodontal stages. This effect was determined by comparative results. A calcium compound and/or a zirconium compound are components of so-called sealers-especially biosealers, which are used to seal a root canal during root canal treatment. The sealer is used to seal the root canal and simultaneously inhibits the focus of inflammation, i.e. the apical whitening 32 in FIG. 2, and induces healing.

The combination of such a biosealer containing a calcium compound and/or a zirconium compound with hyaluronic acid has led to the extremely surprising results of potentiating the bone-forming effect of hyaluronic acid.

For the test results, the respective patient was first subjected to a standard root canal treatment. The treated root canal(s) were sealed with a biosealer containing a calcium compound and/or a zirconium compound. The BioRoot™ RCS biosealer from Septodont containing tricalcium silicate, zirconium oxide and povidone was used for this purpose. Immediately afterwards, the patient underwent the treatment with hyaluronic acid described above, with the difference that only a second measurement was carried out after 4 months. The corresponding results are shown in Table 2.

TABLE 2

| Tooth FDI | Measurement 1 | Measurement 2 |
|---|---|---|
| 47 mesial | 17 mm | <3 mm |
| 47 distal | 10 mm | <3 mm |
| 47 mesial | 25 mm | 8 mm |
| 47 distal | 20 mm | 7 mm |
| 34 mesial | 4.3 mm | <3 mm |
| 34 distal | 7 mm | <3 mm |
| 36 mesial | 11 mm | 3 mm |
| 12 mesial | 13.4 mm | <3 mm |
| 12 distal | 11.7 mm | <3 mm |
| 42 mesial | 12 mm | <3 mm |
| 42 distal | 13 mm | <3 mm |

The results show that by adding a calcium compound and/or a zirconium compound to hyaluronic acid in the treatment of bone formation, in 8 out of 11 examinations the bone formation was so strong that the periodontal gap closed to a level that could be considered healthy. Even in cases where a periodontal gap still remained with a residual size, the growth was considerable compared to conventional treatment with hyaluronic acid.

1. Dental growth stimulant for regenerating a bone structure, in particular within a tooth pocket, characterized in that the growth stimulant comprises at least one hyaluronic acid or a hyaluronic acid derivative, in particular a hyaluronic acid salt, and wherein the growth stimulant further comprises a calcium compound and/or a zirconium compound.

2. Dental growth stimulant according to claim 1, characterized in that the growth stimulant comprises the hyaluronic acid or the hyaluronic acid derivative, in particular the hyaluronic acid salt, and the calcium compound and/or the zirconium compound.

3. Dental growth stimulant according to claim 1 or 2, characterized in that the calcium compound is formed as a silicate compound, in particular as a tricalcium silicate.

4. Dental growth stimulant according to one of the preceding claims, characterized in that the zirconium compound is realized in the form of zirconium (IV) oxide.

5. Dental growth stimulant according to any one of the preceding claims for use in a method of treating paradontosis and/or forming a bone by stimulating the bone in a tooth pocket comprising the steps of:
A performing a root canal treatment of a tooth with the hyaluronic acid or the hyaluronic acid derivative, in particular the hyaluronic acid salt, and
B dispensing the growth stimulant into a periodontal gap between the tooth and a jaw in which the tooth is located.

6. Dental growth stimulant according to claim 5, characterized in that an anesthetic, in particular a local anesthetic, is applied before the root canal treatment.

7. Dental growth stimulant according to one of the preceding claims, characterized in that the growth stimulant is multicomponent and is formed by adding the individual components in situ at the site of application.

8. Treatment set comprising a plurality of storage packages for carrying out various steps of a dental treatment for bone formation and/or for the treatment of paradontosis, characterized in that the treatment set contains at least one storage package with the dental growth stimulant according to one of the preceding claims or storage packages with a plurality of individual components for the in situ preparation of the dental growth stimulant according to one of the preceding claims.

9. Treatment set according to any one of the preceding claims, characterized in that the treatment set comprises a storage package of a local anesthetic.

10. Treatment set according to one of the preceding claims, characterized in that the treatment set comprises a packaging housing, wherein the dosing packages are arranged in individual sterile packages in the packaging housing.
